(19) United States
(12) Patent Application Publication
(10) Pub. No.: US 2025/0239376 A1
(43) Pub. Date: Jul. 24, 2025

(54) HOSPITAL PATIENT COMMUNICATION SYSTEM FOR HALLWAY BEDS WITH INTEGRATED EMERGENCY ALERTS AND TWO-WAY VOICE COMMUNICATION

(71) Applicant: OLINCIA WHITE, Sacramento, CA (US)

(72) Inventor: OLINCIA WHITE, Sacramento, CA (US)

(73) Assignees: OLINCIA WHITE, Sacramento, CA (US); Vital Tech Medical Solutions, LLC, SAN FRANCISCO, CA (US)

(21) Appl. No.: 19/172,660

(22) Filed: Apr. 8, 2025

(51) Int. Cl. G16H 80/00; G16H 15/00; G16H 40/20; G16H 40/40; G16H 40/67

(52) U.S. Cl. CPC G16H 80/00; G16H 15/00; G16H 40/20; G16H 40/40; G16H 40/67

(57) ABSTRACT

A wireless patient communication device, disclosed for hospital emergency departments, specifically for patients in hallway beds. The device enables direct communication between patients and hospital staff through a built-in speaker and microphone. When patients press the call button, the device initiates a call to a pre-programmed recipient, allowing two-way communication. An indicator light illuminates when calls are placed, remaining on until manually turned off, ensuring staff awareness. The device includes emergency buttons: A Code Blue button, which activates a flashing blue light and an emergency alert, and a Rapid Response button, which triggers a flashing red light. A swappable battery ensures uninterrupted operation, while a metal clamp allows attachment to hospital beds or IV poles. The system integrates with a central database for monitoring and logging alerts, improving patient safety and hospital efficiency.

### FIELD OF THE INVENTION

The present invention relates to healthcare communication systems, specifically a wireless communication device designed to enhance emergency and non-emergency communication between patients and healthcare staff in hospital settings. More particularly, the invention provides a system that allows patients, particularly those located in hallway beds or other non-traditional locations, to easily communicate with healthcare personnel through a portable, wireless device with integrated visual and audio features. The system improves the response time for medical assistance, increases patient safety, and streamlines the workflow of healthcare staff.

### BACKGROUND OF THE INVENTION

Hospitals, especially emergency departments (EDs), are fast-paced environments where patient safety, prompt medical attention, and efficient communication are vital. The growing number of patients in need of immediate care often leads to overcrowded hospital settings, especially in emergency rooms where many patients are placed in hallway beds. Although these areas are meant to be temporary, they present a unique challenge for patient care, as hallway patients have no direct or easy way to contact medical personnel in times of distress.

Traditional hospital calls light systems, which allow patients to call for help with the push of a button, are generally limited to rooms where permanent beds and infrastructure are in place. These systems are not designed to accommodate overflow situations in hallways, often leaving patients at risk for delayed care, increased fall incidents, and missed medical emergencies. When hallway patients need assistance, they must rely on verbal calls or hope that a staff member is within earshot or passing by. This system's inefficiency can result in prolonged response times, causing increased frustration for patients and added stress for hospital staff.

### Challenges in Emergency Department Communication Systems

In the fast-paced environment of an emergency department, timely and accurate communication is paramount. This ensures that patients receive care when needed and that staff is properly alerted to assist. However, the traditional call light system, while effective in rooms, does not account for hallway patients, leading to significant gaps in communication. This challenge is compounded by the following issues:

Emergency departments are often overcrowded, and it is not uncommon for patients to be placed in hallway beds. When this occurs, these patients have no dedicated means to communicate with the nursing staff or clerks overseeing the emergency room. Existing call light systems are restricted to patient rooms, and the design of these systems does not include hallway accessibility. In situations where hallway patients cannot press a call button, staff maybe unaware of urgent needs.

Studies have shown that delayed medical response is one of the leading causes of medical complications in hospital emergency settings. Specifically, hallway patients who cannot easily signal for help may experience increased wait times for assistance, leading to critical delays in their treatment. Furthermore, these patients may be at risk of falling or worsening medical conditions if assistance is not provided promptly.

The emergency department's medical staff, including nurses and clerks, are often overwhelmed with patient care tasks. Without an efficient communication system in place, these staff members must constantly monitor hallway patients, which detracts from their ability to attend to other high-priority cases. As a result, hallway patients are not provided with the necessary attention, contributing to delayed care and increased stress for medical staff.

In critical situations, such as when a patient experiences a cardiac arrest or severe respiratory distress, any delay in accessing timely medical care can be fatal. Traditional systems fail to adequately address urgent emergencies that require an immediate response, such as Code Blue (for life-threatening situations) or Rapid Response calls, which are not clearly distinguishable from standard calls for assistance in the existing hospital systems. Emergency buttons for situations like Code Blue are seldom implemented in the context of hallway bed patients, thus missing an essential component for critical care.

The development of a wireless communication device for patients in hallway beds is not entirely novel, as some prior art attempts to solve related problems, but each of these systems falls short in addressing the unique needs of overflow patients in the emergency department.

U.S. Patent Application Publication No. 2024/0038035, discloses a wireless patient call system that allows patients to communicate with medical staff. The system uses a portable device that places calls when a button is pressed. While the system is wireless, it primarily focuses on patient rooms and relies on existing hospital infrastructure to support its operation. Furthermore, it does not include the feature of a persistent light that remains on until manually turned off Additionally, this system lacks the ability to prioritize urgent calls, such as Code Blue or Rapid Response, and does not address hallway patients specifically.

Patent Application Publication No. KR20110099152, discloses a wireless medical communication device that enables patients to contact hospital staff by pressing a button. However, this system suffers from limited scope-it is primarily designed for patient rooms and does not accommodate the specific needs of hallway patients. Additionally, this patent does not provide a visual indicator that persists after the call is initiated, and it lacks distinct emergency response features for situations such as Code Blue.

In a study on hospital call systems, CARECOM developed to keep nurses happy and patients safe, it was emphasized that hallway patients remain underserved by current technologies. The study revealed that patients in hallway beds must rely on verbal calls for assistance, which are often not heard or responded to in a timely manner. Furthermore, the research indicated that existing systems do not offer visual alerts or a way for medical staff to know when hallway patients need help, which is especially important in high-traffic areas like emergency departments.

An article on Nurse Call System: Transform Patient Care with Iota Solutions suggested that colored lights (such as red or blue) could improve hospital communication by providing a visual cue for urgent situations. However, the study acknowledged that many current systems do not implement this technology effectively in hallway or temporary bed assignments. Moreover, manual reset of visual alerts is often overlooked, leading to confusion when multiple alerts are triggered simultaneously.

The present invention proposes a wireless communication device that provides a simple and effective means for hallway patients to request assistance. The device features a two-way communication system that allows patients to talk to hospital clerks or nurses in real time via a built-in speaker and microphone. Upon pressing a button, the device triggers a visual indicator that stays illuminated until manually turned off by the medical staff. The system also includes specialized emergency buttons for Code Blue and Rapid Response, which alert staff to the urgency of the situation through flashing lights and distinct calls.

The device is portable, making it easy for hospital staff to attach it to hospital beds, gurneys, or IV poles using a holding clip. It uses a hot-swappable battery for continuous operation, and its communication portal integrates with hospital systems to provide real-time tracking of all calls for help. This solution addresses the gaps left by traditional call light systems and is designed specifically to improve patient safety, reduce response times, and enhance hospital efficiency.

### SUMMARY OF THE INVENTION

The present invention relates to a wireless communication system specifically designed for use in hospital environments where patients might be in temporary beds, hallway beds, or emergency room (ER) situations. This system aims to address the communication challenges between patients and medical staff, particularly when patients do not have easy access to traditional call systems found in designated patient rooms.

The invention provides an integrated communication tool that is not only wireless and portable but also simple to operate, ensuring immediate and efficient communication with medical staff during emergencies or routine requests. By incorporating, but not limited to a communication module with a button-activated call system, two-way speakerphone communication, a visual indicator light, and specialized emergency buttons, the invention ensures that patients in overflow or hallway beds can easily request assistance, with clear alerts sent to the appropriate medical staff for timely response.

This invention is particularly valuable in emergency settings where quick access to medical personnel can mean the difference between life and death. Additionally, it enhances hospital workflow, increases operational efficiency, and improves overall patient experience.

The heart of the system is the communication module, which enables the device to place calls and transmit signals wirelessly. This module is designed to connect to the hospital's central communication infrastructure (e.g., nurse call systems, electronic health records). When activated, the communication module transmits a signal to alert medical personnel, ensuring the patient's request is logged and the staff is aware of the situation.

The communication module can use Wi-Fi, Bluetooth, or RF communication protocols, making it flexible in terms of hospital network architecture. Its integration with hospital databases means it can trigger alerts, update patient status, and inform staff about ongoing requests, all in real-time.

The primary method by which the patient initiates communication is via the call button. This button can be placed directly on the patient's bed or attached to a flexible cord that the patient can hold or clip onto their clothing. Once pressed, the button sends a wireless signal to the communication module, indicating that the patient requires assistance.

The design of the call button is intuitive, ensuring ease of use for patients with varying levels of mobility or cognitive ability. The button is large enough to be pressed easily by patients in a variety of positions, including those who may be bedridden, elderly, or physically challenged. The device can also accommodate multiple buttons-including the standard call button, along with specialized buttons for emergency situations like Code Blue and Rapid Response.

The device is equipped with a speakerphone system to enable two-way communication between the patient and medical staff. This feature is essential for situations where the patient cannot physically interact with the device but needs to explain their condition. The speakerphone allows for a real-time conversation between the patient and hospital staff, allowing the latter to assess the urgency of the situation, offer instructions, or prioritize medical intervention.

The microphone and speaker system are designed to be high-quality, ensuring clear communication even in noisy hospital environments. The microphone is sensitive enough to pickup the patient's voice clearly, while the speaker is loud enough to be heard over ambient hospital noise. This feature improves the patient's ability to communicate vital information, such as pain levels, symptoms, or medical history, directly to the attending medical staff.

An innovative feature of the invention is the indicator light. When a call is initiated, a visual alert is activated. The light remains on until it is manually turned off by a member of the medical team, indicating that the request for assistance has been received and is being processed. This persistent visual indicator makes it easier for hospital staff to see if any patient requires attention, even if they are not in the immediate vicinity.

This indicator light provides an extra level of security, especially in high-traffic hospital environments where traditional call systems might go unnoticed or where staff cannot always hear auditory signals. The light can be placed on a variety of devices and is designed to remain visible from a distance, ensuring that staff are alerted to a patient's request no matter where they are in the hospital.

The device is equipped with dedicated emergency buttons designed for urgent situations like Code Blue (cardiac or respiratory arrest) or Rapid Response (for patients exhibiting deteriorating conditions). These buttons are easy to access and clearly marked, allowing patients to quickly signal life-threatening situations.

Pressing one of these emergency buttons sends an immediate alert to hospital staff with a higher priority than the regular call button, ensuring that critical situations are escalated appropriately. These emergency signals trigger special visual and audio alerts in the central hospital system, ensuring that medical teams are mobilized without delay.

The device operates on a rechargeable battery housed in a battery compartment. The power source is designed for long-term use, providing sufficient charge for extended periods without frequent recharging. This is particularly important in emergency room environments, where the device must function continuously, even during long shifts or busy hospital hours.

Additionally, the device is equipped with a low-battery warning system to alert medical staff when the battery is running low. This ensures that the device is always operational and ready for use when needed.

The device is designed to be portable, allowing it to be used in a variety of hospital settings. It can be mounted on a hospital bed, clipped onto a gurney, or placed on medical equipment such as IV poles or monitoring systems. This versatility ensures that patients can use the device in overflow beds, temporary beds, or emergency treatment areas where traditional call systems may not be available.

The mounting options make the device easy to use without requiring additional setup, reducing the burden on hospital staff and ensuring seamless integration into existing healthcare infrastructure. The design is intended to ensure that patients in any hospital location can use the device without confusion or difficulty.

One of the most powerful features of this invention is the centralized communication portal. This portal is integrated with the hospital's existing communication systems, allowing staff to manage all incoming calls, prioritize them based on urgency, and track responses in real time.

The portal enables the hospital to maintain an accurate record of patient calls and responses, helping to optimize resource allocation and improve patient care coordination. Staff can access the portal from any device, including smartphones, tablets, or hospital workstations, making it easy to respond to requests no matter where they are located.

The invention is designed to seamlessly integrate with a hospital's existing electronic health records (EHR) and nurse call systems. This means that when a call is made, it not only triggers an alert in the central system but also logs the request in the patient's medical record. This integration ensures that staff have access to relevant patient information in real time, allowing them to make informed decisions quickly.

Additionally, the integration with nurse call systems allows hospital staff to prioritize calls based on the patient's condition, ensuring that those requiring urgent medical intervention receive timely attention. The device is thus a vital tool in enhancing the efficiency and effectiveness of hospital operations, improving both patient care and the hospital's ability to manage high patient volumes.

Several other features can be incorporated into the device, depending on hospital needs. For example, the device can include a display screen to show important information such as the patient's name, the status of the call, or a brief description of the medical condition. This allows staff to quickly assess the situation before responding.

The system can also be upgraded with additional sensors, but not limited to, such as motion detectors or vital sign monitors, that provide real-time data to medical staff. For example, if a patient in distress presses the emergency button, the system could automatically send alerts that include the patient's heart rate, blood pressure, or oxygen saturation, enabling medical staff to take swift action.

### Advantages of the Invention

The wireless communication tool significantly improves patient safety by providing a reliable and efficient means for patients to request help. The persistent indicator light ensures that medical staff are always aware of the patient's request, even when they are not within earshot. This leads to faster responses and fewer missed calls.

By integrating with existing hospital systems, the device streamlines workflow by ensuring that requests are automatically logged, prioritized, and tracked. This integration helps hospitals manage patient care more effectively, reducing the risk of errors and delays.

The wireless communication tool is highly scalable and can be deployed across hospitals of any size. Its portability ensures that it can be used in overflow areas, while its ability to integrate with existing systems makes it a cost-effective solution for improving hospital communication.

The ability to make an instant connection with the medical staff and provide real-time information reduces response time, leading to improved patient outcomes. The two-way communication system allows staff to assess the situation remotely, potentially taking action without needing to be physically present.

This wireless communication tool offers a comprehensive solution to the problem of communication breakdowns in hospital settings, particularly for patients in temporary beds or hallway beds. Its ability to improve patient safety, streamline hospital workflows, and enhance emergency response makes it an essential device for modern healthcare environments. The system's flexibility, integration capabilities, and ease of use ensure that it can meet the needs of hospitals, whether in large urban centers or smaller community-based facilities. By providing a portable and reliable communication tool, the invention has the potential to revolutionize how hospitals manage patient care, particularly in times of high demand and critical need.

### BRIEF DESCRIPTION OF THE ACCOMPANYING DRAWINGS

The following description should be read with reference to the drawings. The drawings, which are not necessarily to scale, depict examples and are not intended to limit the scope of the disclosure. The disclosure may be more completely understood in consideration of the following description with respect to various examples in connection with the accompanying drawings, in which:

FIG. 1 is a perspective view of the wireless call light device (100);

FIG. 2 is a front elevation view of the wireless call light device (100) showing the indicator light (102) and patient call button (106);

FIG. 3 is a rear elevation view of the wireless call light device (100) showing the staff-accessible buttons;

FIG. 4 is a side elevation view of the wireless call light device (100) illustrating the grip (118) and battery compartment (120);

FIG. 5 is a block diagram of the primary components of the wireless call light device (100) are portable electronic device (122), including memory (124), processor (126), communication portal (128), and power source (130);

FIG. 6 is a detailed view of the device (100) attached to an IV pole using the holding clip (132) and showing the rod (134);

FIG. 7 is a close-up view of the patient-accessible buttons, specifically the patient call button (106) and speaker (108);

FIG. 8A and FIG. 8B are a close-up view of the staff-accessible buttons on the rear of the device and patient-accessible buttons;

FIG. 9 is illustrating the diagrams of wireless call light device (100), which mechanically coupled to a patient bed in accordance with exemplary embodiments of the present invention;

FIG. 10 is a block diagram illustrating a wireless call light devices communication with the central communication module according to the present invention;

FIG. 11 is a detailed block diagram of the device's electronic components, including the microphone (144), audio interface (146), speaker (108), radio (148), and DSP (150);

FIG. 12 is a flowchart illustrating the device's operational sequence;

FIG. 13 is a flowchart illustrate the sequence of steps involved when a patient presses the call button on the device, the communication that takes place between the patient and the healthcare staff, and the resulting actions; and

FIG. 14 is a flowchart describe the steps involved when staff members interact with the device for critical scenarios such as a Code Blue or Rapid Response situation.

### DETAILED DESCRIPTION OF THE INVENTION

The invention addresses a significant gap in the existing communication infrastructure within hospital emergency departments (ED) and similar healthcare settings. Specifically, it focuses on the wireless communication system that enables patients in hallway beds or non-traditional locations to have direct, real-time communication with medical staff. The system comprises a patient call button, a central communication portal, and several features designed to ensure timely assistance in emergency situations, including visual indicators, audio interfaces, and manual override functions.

The device is designed to improve the workflow of the medical staff, enhance patient safety, and ensure efficient communication in situations where traditional call light systems may not be adequate, particularly in hallway settings.

The wireless communication system consists of several key components that work together seamlessly to ensure reliable and effective communication between the patient and the hospital staff.

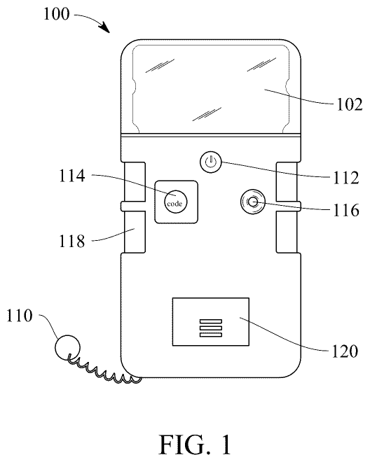

FIG. 1 illustrates an embodiment of the wireless call light device (100) in a perspective view. The device includes an enclosure (104) that houses the internal components and provides structural integrity. The enclosure (104) includes an indicator light (102) is positioned on the front face of the device, serving as a visual alert when the patient initiates a call. Further, the enclosure (104) also includes a patient call button (106) is mounted on the device, allowing patients to request assistance. The speaker (108) is integrated into the enclosure to enable two-way audio communication between the patient and hospital staff. This embodiment provides an overall view of the major external elements of the device.

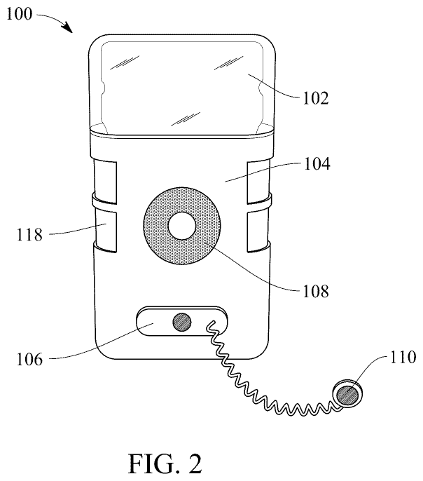

FIG. 2 presents a front view of the device (100), showing key patient-accessible features. The indicator light (102) is positioned prominently to provide a clear visual signal when activated. The patient call button (106) is placed ergonomically to be easily accessible for patients. The speaker (108) is located in the enclosure to facilitate audio interaction between the patient and medical personnel. This embodiment ensures an intuitive user interface, making it easy for patients to operate the device.

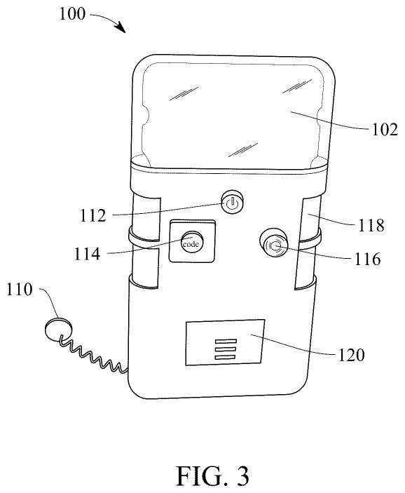

FIG. 3 illustrates the rear view of the device (100), where staff-accessible buttons are located. This embodiment includes a power button (112) to turn the device on and off. A code blue button (114) is incorporated for emergency situations requiring immediate intervention. Similarly, a rapid response button (116) is provided to enable urgent medical alerts. These buttons are positioned on the back of the device to prevent accidental activation by the patient while ensuring easy accessibility for medical personnel.

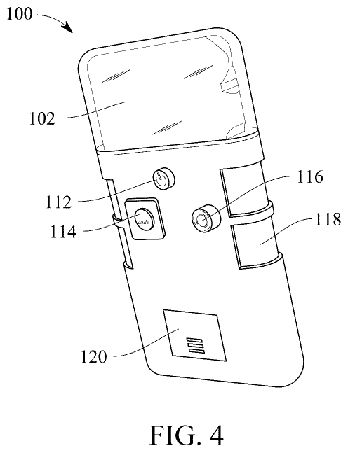

FIG. 4 depicts a side view of the device (100), highlighting the grip (118) and battery compartment (120). The grip (118) ensures secure handling, preventing accidental falls or displacements. The battery compartment (120) is designed for easy battery replacement and supports a hot-swappable battery (130) to ensure uninterrupted operation. This embodiment showcases the ergonomic design of the device, making it convenient for both patients and healthcare providers.

The indicator light (102) is a crucial element of the invention, serving both as a visual alert for the medical staff and as a confirmation of an active communication session. The light is activated whenever the patient call button (106) is pressed. The light remains illuminated until manually turned off by a staff member, ensuring that even if the clerk or nurse is otherwise occupied, another staff member passing by the patient can quickly identify the need for assistance. Additionally, the indicator light can have a color-coded system to signify the urgency of the request (e.g., yellow for normal, red for urgent, blue for Code Blue, and flashing red for Rapid Response).

The enclosure or case of the device (104) houses all the internal components of the system, providing protection and facilitating ease of use. It is designed to be robust, yet lightweight, with an ergonomic shape that allows it to be easily attached to a hospital bed or IV pole using a clamp. The enclosure also includes a battery compartment (120) to house the swappable battery, ensuring continuous operation throughout the day. It also houses the communication module, which is responsible for sending and receiving signals between the patient device and the hospital communication system.

The patient call button (106) is the primary mechanism for the patient to request assistance. This button is located on the device's body or mounted on a flexible cord (110), which is typically attached to a wand that the patient can easily hold. When the button is pressed, it immediately sends a signal to the hospital communication system to initiate the call. This button may also include a light to indicate that the signal has been successfully sent.

The speaker (108) is embedded within the device to allow two-way communication between the patient and the hospital staff. It functions alongside the microphone (144), creating a speakerphone-like feature that enables real-time communication. The patient can speak to the medical staff via the speaker, and the staff can respond to the patient through the same interface.

A Code Blue button (114) is included as an emergency feature that allows the patient or staff to signal a life-threatening emergency. When pressed, the light on the device will flash blue, and a signal will be sent to the hospital's central communication portal, indicating that immediate action is required. This functionality ensures that medical personnel can respond to critical situations as quickly as possible.

Similarly, the Rapid Response button (116) serves to indicate an urgent situation requiring immediate attention. When pressed, the device's light will flash red, and a signal will be sent to alert the hospital staff. This button is designed for less critical, but still urgent, medical situations that require swift intervention.

The grip (118) is incorporated into the design of the device to make it easier for staff to handle and move the device when required. It is typically located on the back or side of the unit and is ergonomically positioned to allow a comfortable and firm hold.

The device is powered by a hot-swappable battery stored in the battery compartment (120). The swappable feature allows the device to be easily recharged or replaced without requiring the entire system to be taken out of service. A charging base is included to allow the device to be placed for charging when not in use.

The wireless call light device (100) includes a portable electronic device (122) that refers to the entirety of the wireless communication system, designed to be mobile and easy to transport within a hospital environment. The device is lightweight, compact, and can be clipped to various locations near the patient, including IV poles, gurneys, or other patient-supporting equipment.

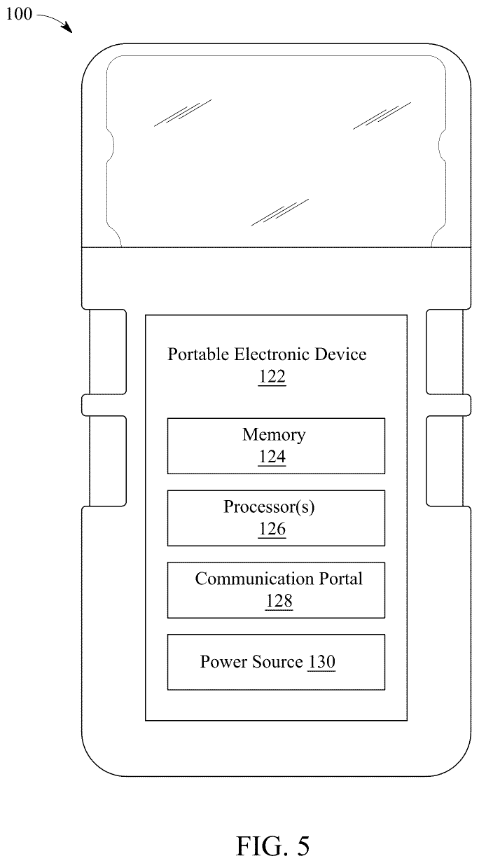

FIG. 5 illustrates a schematic representation of the internal components of the wireless call light device (100). The wireless call light device (100) includes a portable electronic device (122). Further, the portable electronic device (122) includes a memory (124), a processor (126), a communication portal (128) and power source (130). The memory (124) stores configuration settings and operational data. The processor (126) controls various functions of the device, including signal processing and communication. The communication portal (128) facilitates data transmission between the device and hospital systems. The power source (130) provides energy for continuous operation. This embodiment details the electronic architecture of the device.

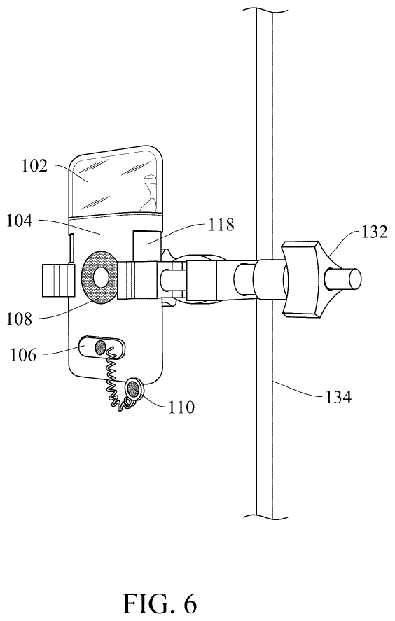

FIG. 6 illustrates how the device (100) is securely attached to an IV pole using a holding clip (132). The rod (134) provides additional support, ensuring stability when mounted on hospital equipment. This embodiment demonstrates the adaptability of the device for different hospital settings, allowing easy placement in various patient care environments.

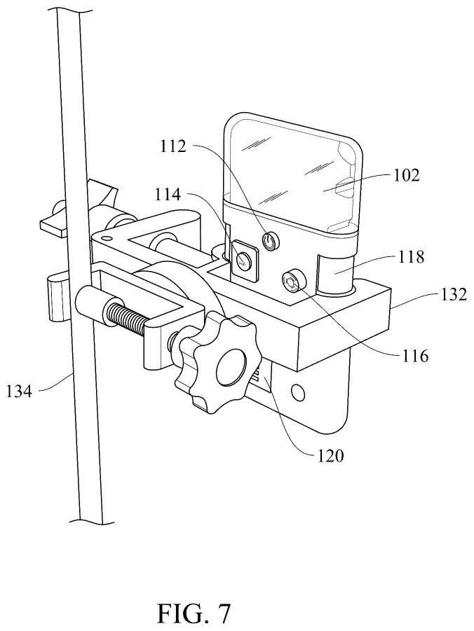

FIG. 7 illustrates a detailed view of the patient-accessible controls. The patient call button (106) is designed to be easily pressed by the patient when assistance is needed. The speaker (108) is integrated into the device to enable real-time voice communication. This embodiment highlights how the device ensures seamless patient-to-staff interaction.

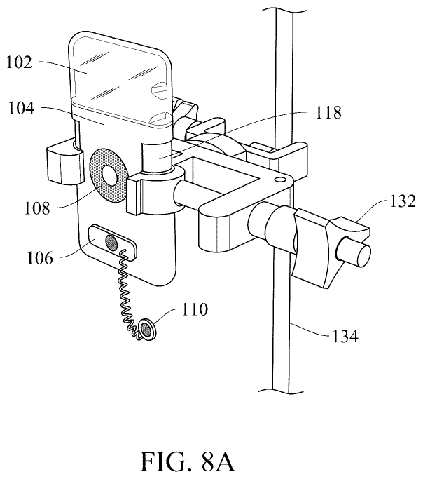

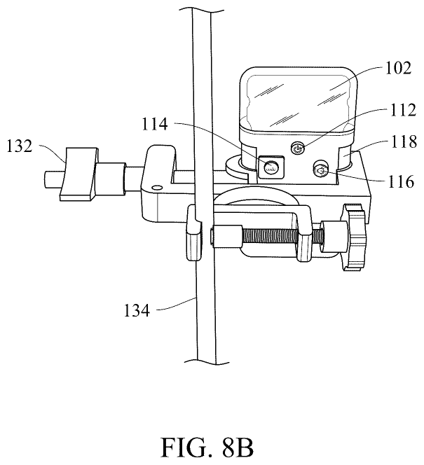

FIG. 8A and 8B focuses on the emergency response features of the device (100). The code blue button (114) is positioned to allow medical staff to activate a critical alert when an emergency occurs. The rapid response button (116) serves as a secondary alert system for urgent but non-life-threatening situations. This embodiment enhances hospital emergency protocols by providing a quick and efficient alert mechanism. The power button (112) located on the back of the device. This button allows hospital personnel to manually tum the device on or off. This embodiment ensures that the device can be quickly powered on or reset as needed.

The battery charging base, which is designed to accommodate multiple devices simultaneously. The charging base ensures that spare batteries remain fully charged and ready for use, allowing seamless operation without interruptions. This embodiment supports continuous device availability in busy hospital environments.

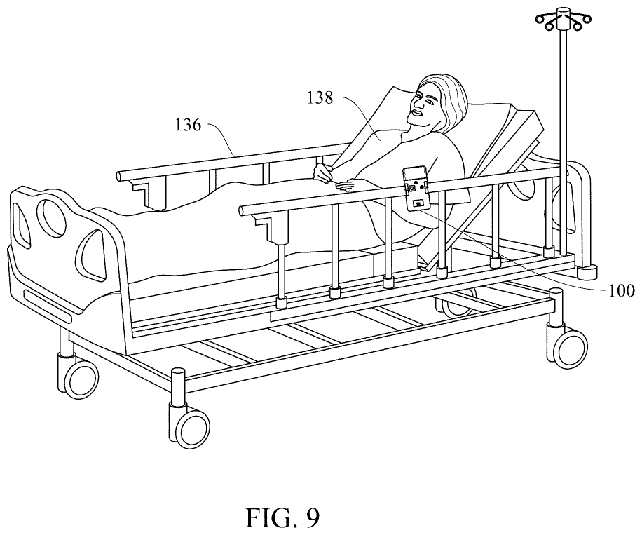

FIG. 9 illustrates the wireless call light device (100), in embodiments, may be mechanically coupled to, but not limited one or more patient bed (138). For example, as shown in FIG. 6, the device (100) may include the holding clip (132). The holding clip (132), in embodiments, may attach to, but not limited an IV pole or rod (136). The device 100 may be mechanically coupled to a wheelchair and/or a bed.

The adjustable clamp mechanism used to secure the device (100) to various hospital equipment. The clamp ensures stability while allowing easy repositioning. This embodiment enhances the versatility of the device by enabling attachment to different structures such as hospital beds, IV poles, and medical carts.

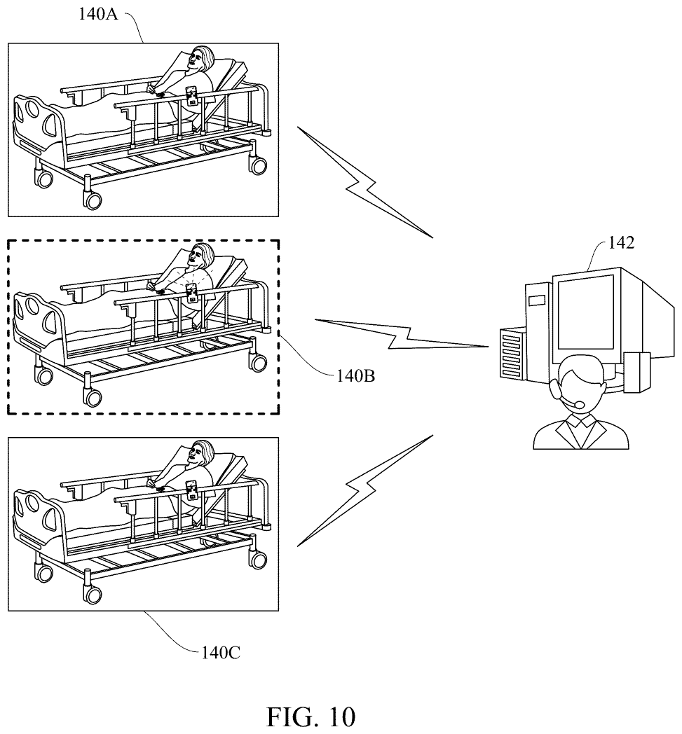

FIG. 10 is a block diagram illustrating a wireless call light device according to the present invention. This device is designed to enable wireless communication within a hospital and assigns an ID to each bed (140A-140C) for each floor and/or room arranged in a ward. When a call is made via the wireless button (106), the call signal is received through the central communication module (142), and the module displays the entire ward and highlights the bed (140B) that sent the call signal on the monitor. The module includes a main server, typically installed in a location such as the nurse's room, which is configured to transmit the call signal—formatted as a text message—to the nurse and/or doctor in charge, based on the ID of the call signal.

The registration process for the device (100) when assigned to a patient. When a nurse retrieves the device, the power it on using the power button (112). The device prompts for an assigned bed number, which is input via a voice system. Once the device is successfully registered, it announces its identification, such as “Hallway 5,” and is ready for use. This embodiment details the ease of setting up and configuring the device in a hospital environment.

The call signal's ID is unique for each device and each bed (140A-140C) within the room, allowing the central communication module (142) to identify the room and bed (140A-140C) when the call signal is received. This feature enables nurses and doctors to immediately identify the calling patient, thereby facilitating prompt treatment.

The central communication module (142) is located in the nurse's room, is programmed to display a pop-up window on the monitor according to the call signal. It shows the ward number, bed position (such as patient name), and other details based on the ID of the incoming call.

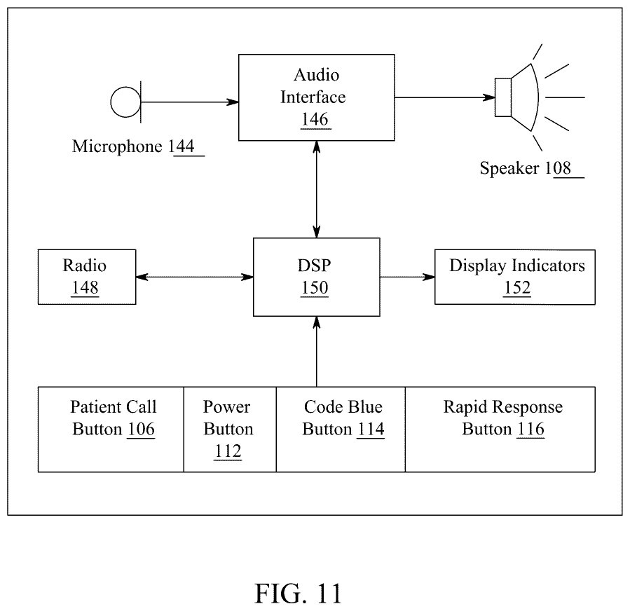

FIG. 11 illustrates a comprehensive diagram of the electronic systems integrated into the device (100). The microphone (144) captures patient audio input. The audio interface (146) processes voice signals for communication clarity. The speaker (108) outputs the staff's response. The radio (148) facilitates wireless data transmission, while the digital signal processor (DSP) (150) optimizes audio quality. This embodiment provides a technical overview of how different electronic components interact within the system.

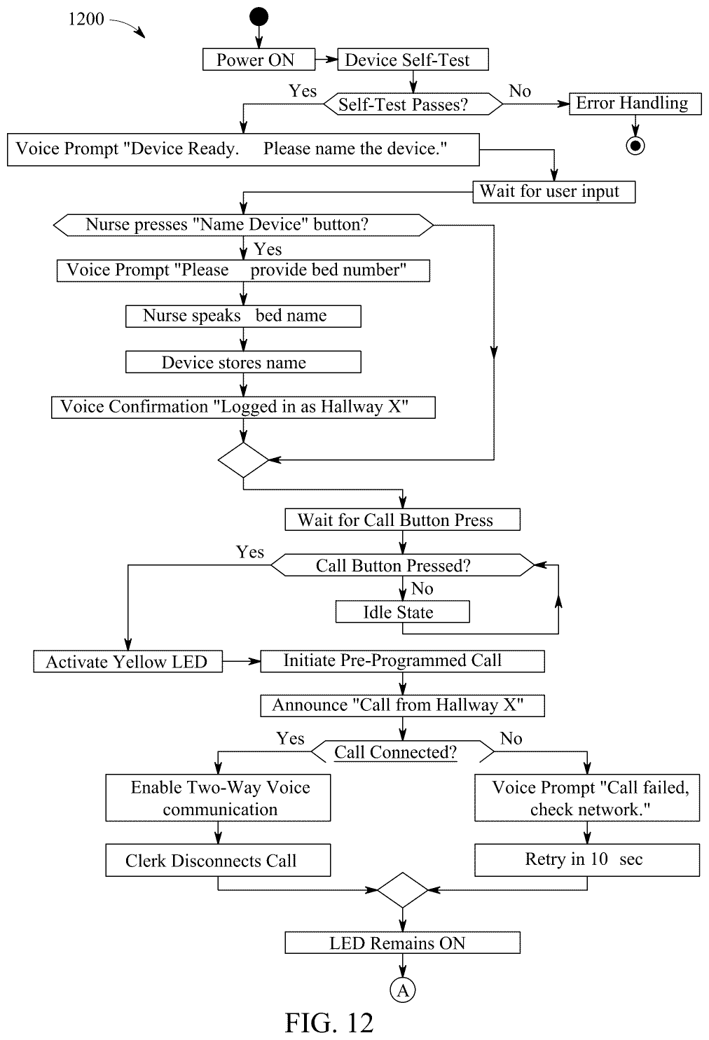

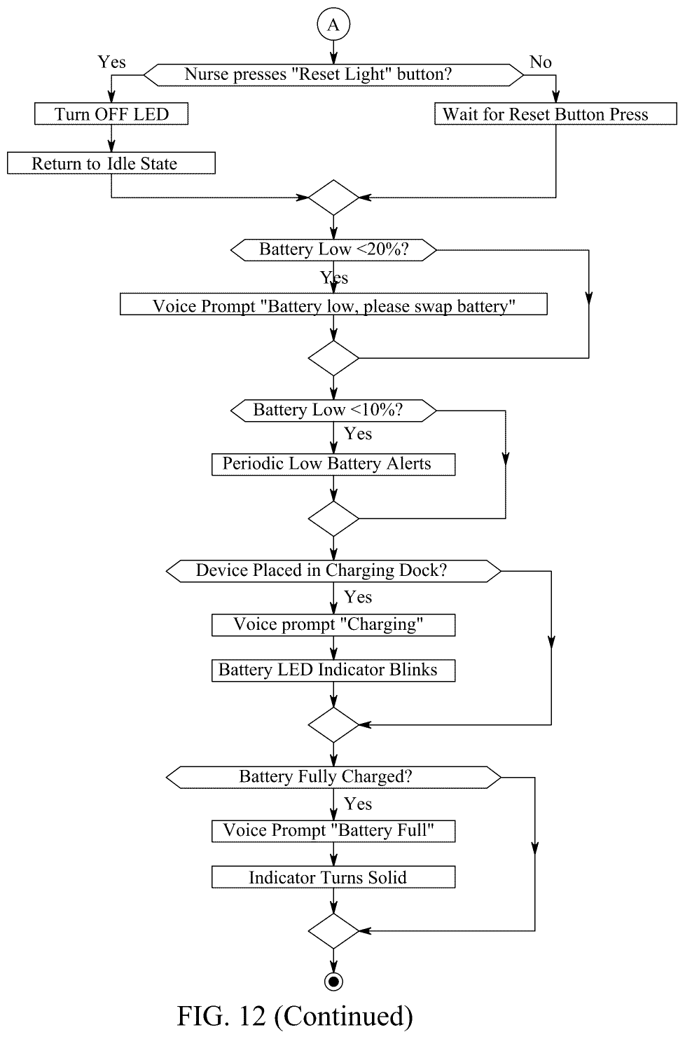

FIG. 12 illustrates a process flow for how the device (100) operates. When the patient call button (106) is pressed, a signal is sent via the communication portal (128) to the hospital's central system. The indicator light (102) is activated, and an audio connection is established via the speaker (108). The staff member at the central system responds through the device, enabling real-time communication. The light remains on until manually turned off by medical personnel. This embodiment illustrates the step-by-step process of how the system functions in a hospital setting.

When the device is initially set up, it must be powered on by the medical staff using the power button. The device will emit a confirmation sound or indicator light to indicate that it is ready for use.

The nurse or medical staff will then assign a name to the device, corresponding to the patient's bed number or location (e.g., Hallway 5, Room 103, etc.). This step ensures that when the call is received, the staff member knows exactly where the call is coming from.

The device is equipped with an adjustable clamp or mounting bracket, allowing the staff to attach the device securely to the hospital bed, IV pole, or gurney. The flexible cord attached to the patient's call button allows the patient to have easy access to the button wherever they are positioned.

The call button can either be directly mounted on the device or placed on the flexible cord for ease of access. The speaker and microphone are built into the device and remain in close proximity to the patient during use.

The patient will press the call button to request assistance. Once the button is pressed, the indicator light on the device will tum on, and a signal will be sent to the communication portal. The light will remain on until it is manually deactivated by a staff member, ensuring that the request for help is visible to those in the vicinity, even if they are not directly near the device.

Simultaneously, the communication portal will display the patient's location, and medical staff will receive the alert. If necessary, the system can also alert specific medical personnel based on the severity of the request (e.g., code blue for emergencies or rapid response for urgent but non-critical issues).

Once the call is received at the communication portal, the medical staff can respond to the patient via the speaker and microphone on the device. This allows for two-way communication, where the healthcare provider can inquire about the patient's needs, provide instructions, or assess the situation.

The volume and clarity of communication are adjustable through the audio interface. Noise-canceling features ensure that the conversation remains clear even in noisy hospital environments.

If the situation is critical, the medical staff can press the code blue button or the rapid response button. This will activate a flashing blue or red light on the device and send an emergency signal to the communication portal, alerting the appropriate team.

The code blue button is specifically designed for emergency situations that require immediate intervention, such as cardiac arrest or other life-threatening conditions. The rapid response button is used for situations that are urgent but do not require the immediate, full-scale response associated with a code blue.

The device includes a battery compartment that must be checked regularly to ensure that the device is powered adequately. The rechargeable battery is designed to last through an entire shift, but staff must be vigilant about checking the battery life to ensure that it remains operational.

When the battery is low, the system will send a notification to the communication portal, alerting the staff to recharge or replace the battery as necessary. By following these steps, the wireless communication system ensures a smooth and efficient process for requesting and providing assistance in a hospital setting. The system is designed to be adaptable, easy to use, and reliable, making it a crucial tool for improving patient care and hospital workflows.

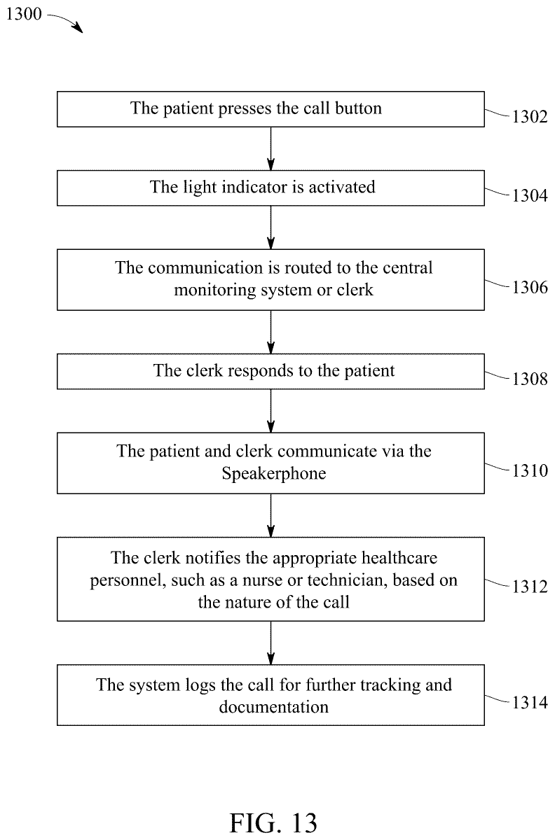

FIG. 13 illustrates two-way communication of the invention. Upon activation of the call button, an LED light is illuminated, signaling that the patient is requesting assistance. The light remains on until manually turned off by a medical staff member. This ensures that the call is not ignored or missed, even if hospital staff is not immediately near the device. The persistent visual indicator makes the communication system more reliable, especially in environments where patients may be out of direct line of sight of staff

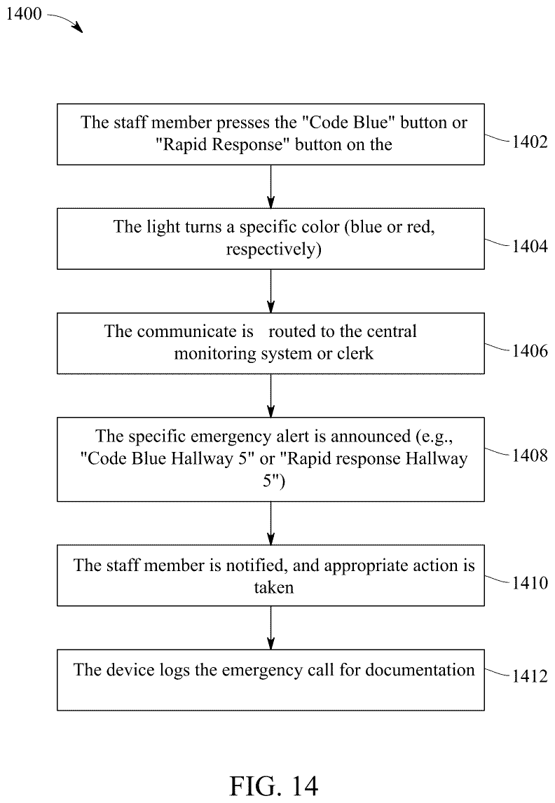

FIG. 14 illustrates a separate flowchart demonstrating how the code blue button (114) and rapid response button (116) function. When a staff member presses the code blue button (114), the device automatically places a high-priority alert to hospital emergency teams. Similarly, pressing the rapid response button (116) sends an urgent but lower-priority alert. This embodiment emphasizes how the device enhances emergency response workflows in hospitals.

From FIG. 12-14, the wireless communication system works by creating a two-way communication channel between the patient and the medical staff. Below is a step-by-step breakdown of how the system operates:

Device Activation Upon patient placement in a hallway bed (136), a nurse or medical staff member assigns the device to the patient's bed. The patient call button (106) is mounted either on the device or on a flexible cord (110) that can be easily placed in the patient's reach. The nurse powers on the device by pressing the power button (112), and the system prompts the nurse to assign the device a name corresponding to the patient's bed number. For example, the nurse could name the device “Hallway 5” for a patient in bed 5. Once the name is confirmed, the device is ready for use.

Patient Call When the patient requires assistance, they simply press the patient call button (106) or the flexible cord button (110). This action sends an alert to the hospital communication system (1302). The alert triggers several actions:

The indicator light (102) turns on, signaling the need for assistance (1304).

A call is placed to the clerk or central desk (via radio (148) or Wi-Fi communication) to notify them of the patient's request (1306).

The patient's name and assigned bed number are announced to the clerk, such as “Call from Hallway 5.”

Communication with Medical Staff Once the call is received, the clerk can communicate with the patient using the built-in speaker (108) and microphone (144) on the device. This allows for clear and immediate communication, without the patient needing to shout or rely on staff hearing their verbal requests. The clerk can then relay the message to the appropriate medical personnel, such as a nurse or technician (1308-1314).

Urgent Requests for emergency situations, the patient or staff can press the Code Blue (114) or Rapid Response (116) buttons (1402). Upon activation:

The light on the device will flash the respective color (blue for Code Blue, red for Rapid Response) (1404).

A call will be made to the clerk desk, and the emergency message will be announced, such as “Code Blue Hallway 5” or “Rapid Response Hallway 5.” (1406-1408)

The staff is immediately made aware of the urgent situation and can proceed with an appropriate response (1410).

After the call has been placed, the indicator light remains on until manually turned off by a staff member. This feature ensures that the request is not overlooked, even if the clerk or nurse is occupied with other tasks. The manual override process is simple, requiring the staff to press the light on/off button located on the device.

The device is powered by a swappable battery located in the battery compartment (120). The swappable battery ensures that the device can be quickly recharged and ready for use. A charging base allows the device to be docked and charged when not in use, ensuring no downtime for critical communication.

### Advantages of the Invention

The wireless communication system offers numerous advantages over traditional patient call systems, particularly in hospital emergency departments or temporary patient beds in hallways.

Increased Safety and Efficiency by enabling wireless communication between the patient and hospital staff, the system reduces response times, ensures that patient requests are not overlooked, and enables medical staff to respond more quickly to urgent needs.

The simplicity of pressing a button to call for assistance and the ease with which the device can be assigned to a patient ensures that it is user-friendly for both patients and staff

The portable nature of the device allows it to be used in temporary bed settings or hallways, which may not be served by traditional call light systems. Additionally, the device can be clipped to various locations within the hospital.

The inclusion of two-way audio communication ensures that patients and medical staff can communicate directly without unnecessary delays or misunderstandings. This is especially important in time-sensitive medical situations.

The ability to call for help quickly and directly reduces patient anxiety and improves their overall experience in the hospital. This invention provides a novel solution to an existing problem in the healthcare industry. By combining wireless communication, visual alerts, and audio interfaces, the device enhances the efficiency and safety of emergency and non-emergency communication in hospitals. The system's user-friendliness, flexibility, and reliability make it an essential tool for improving patient care and staff workflow in healthcare environments.

This invention provides a novel solution to an existing problem in the healthcare industry. By combining wireless communication, visual alerts, and audio interfaces, the device enhances the efficiency and safety of emergency and non--emergency communication in hospitals. The system's user-friendliness, flexibility, and reliability make it an essential tool for improving patient care and staff workflow in healthcare environments.

The wireless communication system addresses a significant need for more effective communication between patients and healthcare providers, particularly in hospitals where rapid responses are often required. In such settings, every second counts when patients need medical attention, and current communication methods, which typically involve wired intercom systems or phone calls, can be inefficient and prone to delays. This invention eliminates those inefficiencies by allowing patients to make a direct and immediate request for assistance through an easy-to-use call button. The system is designed to be intuitive, making it simple for patients to signal for help, regardless of their physical or cognitive limitations.

One of the key features of the invention is the use of an indicator light that provides a visual signal whenever a patient has requested assistance. The light turns on as soon as the call is placed and remains on until manually turned off by the medical staff This feature helps ensure that hospital staff are immediately aware of the patient's request, even if they are engaged with other tasks or located in different areas of the hospital. This continuous visual cue eliminates the need for staff to constantly monitor a screen or a phone for incoming requests, reducing response time and improving the overall workflow.

The system is equipped with multiple patient call buttons, which can be mounted either directly on the device or attached to a flexible cord for easy accessibility. This flexibility ensures that the patient can choose the most comfortable and convenient location for the call button, whether it's attached to the hospital bed or held in hand. When the patient presses the button, a signal is sent wirelessly to the hospital's central communication system, notifying medical staff of the need for assistance. The wireless nature of the system makes it adaptable to different hospital layouts, ensuring that communication can take place from any location within the hospital without the need for physical wiring.

The device's two-way communication feature allows patients to not only request help but also communicate directly with medical staff The system includes a speaker and microphone, enabling the staff to listen to the patient's needs and respond promptly. This audio interface ensures that the patient's concerns are heard clearly, even in noisy environments like hospital hallways or emergency rooms. The system is equipped with noise-canceling technology that filters out background noise, making communication clear and efficient. Additionally, the volume of the speaker can be adjusted automatically based on the surrounding noise levels, ensuring that the patient's voice can always be heard.

To further improve the system's functionality, the invention includes a power button that enables medical staff to turn the device on and off This button is designed to be easy to locate and press, even when the staff is wearing gloves or engaged in other tasks. The power button is an essential feature for ensuring the device operates only when needed, helping to conserve battery life and prevent accidental activations.

The device's battery compartment houses a rechargeable battery that provides continuous power for the system. The battery is designed to last through an entire shift, ensuring that the device remains operational without the need for frequent recharging. When the battery runs low, the system includes an alert mechanism to notify staff that it's time for recharging. Additionally, the device is designed to be easily placed in a charging station when not in use, allowing it to be quickly recharged and ready for the next use. This self-contained power management system eliminates the need for external power sources or complicated setups, making the device fully portable and versatile.

The communication portal serves as the central hub for managing patient requests and directing them to the appropriate medical staff When a call is made by a patient, the communication portal receives the signal and forwards it to the designated staff member, such as a nurse or doctor, who is best equipped to address the patient's needs. The portal operates on a wireless communication network, such as Wi-Fi or radio frequency, ensuring reliable transmission of the patient's request. This system can be integrated into existing hospital infrastructure, seamlessly working with other communication systems, such as nurse call systems or emergency response systems.

To ensure the system is easy to use, the interface is designed with simplicity in mind. The indicator light provides a clear visual cue for staff, while the speaker and microphone allow for clear two-way communication between patients and staff The device can be mounted in various ways, allowing it to be placed near the patient's bed, on a gurney, or on an IV pole. The mounting system includes adjustable clips and brackets, making it easy to position the device in a way that's most convenient for the patient and staff.

The system is also designed for easy maintenance. The battery compartment can be accessed quickly for battery replacement or servicing, and the components are modular, making it easy to repair or replace individual parts. The entire system is built with durability in mind, using high-quality materials that can withstand the rigors of a hospital environment. The components are designed to be easy to clean, ensuring that infection control protocols are met.

In terms of scalability, the system can be expanded to accommodate larger healthcare facilities. Multiple communication portals can be set up across the hospital, ensuring that all patient requests are efficiently routed to the appropriate staff The system is flexible enough to be adapted to various hospital layouts, whether for a small clinic or a large multi-building hospital complex. The wireless nature of the system allows it to be easily expanded without the need for extensive rewiring or infrastructure changes.

Furthermore, the system can be integrated with other hospital systems to improve its functionality. For instance, it can be connected to the hospital's electronic health records (EHR) system, allowing staff to access patient information and medical histories in realtime. This integration can help staff provide more accurate and timely care, as they'll have all the necessary information at their fingertips when responding to a patient's request.

The device also features a manual override function for the indicator light. Once the patient's request is addressed, the medical staff can manually turn off the indicator light, signaling that the issue has been resolved. This simple yet effective feature ensures that staff can maintain control over the system while minimizing the risk of confusion or redundant calls.

The wireless communication system revolutionizes patient care in healthcare environments. By combining visual, audio, and wireless technologies, the device streamlines communication between patients and healthcare providers, improving both the efficiency of staff workflows and the quality of patient care. The system's portability, ease of use, and integration with existing hospital systems make it a versatile and essential tool in modern healthcare. This invention not only enhances patient safety by ensuring timely responses but also helps medical staff manage their tasks more effectively, ultimately leading to better patient outcomes and a more efficient hospital environment.

The device consists of several components that must be assembled carefully to ensure functionality and durability. The key parts of the device are, but not limited to the indicator light, enclosure (case), patient call button, speaker, power button, code blue button, rapid response button, battery compartment, communication module, and communication portal. These components work together to form a complete and integrated system that enables communication between patients and healthcare providers.

The enclosure of the device is the main body, designed to protect the internal components. Itis typically made from durable plastic or other high-quality, medical-grade materials that are easy to clean and resistant to the harsh conditions of a hospital environment, including disinfection agents. The enclosure is designed with a compact form factor to be easily mounted or placed in various positions near the patient's bed or on an IV pole. The device's design incorporates a mounting bracket or clip that allows for secure attachment to a variety of surfaces, ensuring flexibility in deployment.

The indicator light is placed on the front or top of the device for visibility. The light turns on when the patient presses the call button, serving as a visual alert for staff that assistance is required. The light stays on until the medical staff manually deactivates it. This feature ensures that even if a staff member is not immediately available to answer the call, others in the area can see that assistance is needed. The light must be bright enough to be visible even from a distance or in low-light conditions.

The patient call button is located on the front of the device. It is designed to be large and easy to press, ensuring that patients, even those with limited mobility, can easily request assistance. The button is a simple, pressure-sensitive switch that sends a wireless signal to the communication portal when pressed. This button can be mounted on the main device or attached to a flexible cord, allowing the patient to place it wherever is most convenient, such as on the bed frame or IV pole. When the patient presses the button, the call is triggered, sending a signal to the communication portal and activating the indicator light.

The speaker is used for two-way communication between the patient and the staff. It is integrated into the device, with the microphone allowing the patient to speak to the healthcare provider, and the speaker allowing the healthcare provider to respond. The speaker is designed with noise-canceling technology to ensure clear communication in the noisy environment of a hospital. The volume can be adjusted automatically based on ambient noise levels to ensure the patient's voice is audible.

The power button is located on the device's exterior and is used to turn the device on or off. It is designed to be easy to use, even for staff wearing gloves or operating under time constraints. The button is placed in a convenient location, ensuring that staff can activate or deactivate the device without unnecessary effort. When the device is powered on, it is ready to receive calls from patients and communicate with the central communication portal.

The code blue button is a special feature designed for emergencies. This button sends a call to the central desk with the message “Code Blue” followed by the patient's bed number or location. The light on the device will flash blue, and a specific emergency protocol is activated at the healthcare facility. This button is located on the back of the device to prevent accidental activation by patients. The placement of the button ensures that only medical staff can trigger a code blue call.

The rapid response button functions similarly to the code blue button, but it is used in situations that require urgent attention but are not critical emergencies. When pressed, the button sends a signal to the communication portal with the message “Rapid Response” followed by the patient's bed number or location. The light on the device will flash red, signaling that urgent but non-emergency assistance is needed. Like the code blue button, the rapid response button is located on the back of the device, preventing accidental presses by patients.

The battery compartment houses the rechargeable battery that powers the device. The battery should be capable of lasting through a full shift (typically eight hours) without needing to be recharged. The battery is designed to be easily replaceable, ensuring the device remains operational throughout the day. The battery is accessed via a compartment that can be opened for replacement or recharging. The rechargeable battery is capable of retaining power for an extended period, reducing the need for frequent charging.

The communication module is the heart of the device, providing wireless connectivity to the central communication portal. The communication module uses Wi-Fi or other wireless communication protocols (such as Bluetooth or radio frequency) to send signals between the patient's device and the healthcare staff s central communication system. This allows for immediate transmission of the call to the appropriate nurse, doctor, or emergency response team. The wireless nature of the device eliminates the need for complicated wiring, making it adaptable to various hospital layouts.

The communication portal serves as the interface for healthcare providers to receive notifications from the wireless call buttons. When the patient presses a button on the device, the communication portal receives the signal and alerts the designated medical staff The portal is typically located at the nurse's station or other central location within the hospital. The portal can be integrated with other hospital systems, such as the electronic health record (EHR) system or emergency response protocols, allowing staff to access important patient data and provide more accurate and timely care.

In the detailed description, the venous components of the wireless communication system, as shown in the accompanying drawings, are labeled using reference numerals for ease of identification. These reference numerals allow for a direct correlation between the functional description and the physical elements illustrated in the drawings.

Indicator Light (102)—The indicator light serves as a visual alert to signal the status of a call request. It turns on when the patient presses the call button and stays on until manually deactivated by the medical staff. This feature ensures that the healthcare staff is clearly notified when assistance is needed, even if the staff member is not immediately available to respond.

Enclosure/Case (104)—The enclosure, or main body of the device, houses the internal components of the wireless communication system. The enclosure protects the delicate electronics and is designed to withstand the harsh conditions of a hospital environment, such as frequent disinfection, drops, or accidental exposure to liquids.

Patient Call Button (106)—The patient call button is a key element of the system that allows patients to request assistance. Located on the device, it is activated when pressed and sends a signal to the communication portal, triggering the indicator light to tum on. An alternative design allows the patient call button to be mounted on a flexible cord (110), making it more convenient for patients to access from various locations.

Speaker (108)—The speaker is integrated into the device and is used for two-way communication between the patient and the medical staff It allows the healthcare provider to communicate with the patient, responding to their request for help or offering instructions.

Power Button (112)—The power button is used to turn the device on or off This button must be easily accessible to medical staff and is designed to be operable even when staff are wearing gloves or are in a hurry.

Code Blue Button (114)—The code blue button is a specialized feature that allows staff to initiate an emergency alert. When pressed, the button sends an urgent signal to the communication portal and triggers the flashing blue light to indicate that immediate medical assistance is required.

Rapid Response Button (116)—Similar to the code blue button, the rapid response button is used for urgent situations but not for life-threatening emergencies. It triggers a rapid response protocol and flashes a red light, allowing medical staff to respond quickly without the need for a full-scale emergency response.

Grip (118)—The grip is an ergonomic feature designed to make it easier for healthcare providers to hold or mount the device. It provides a secure hold for medical staff when carrying or repositioning the device.

Battery Compartment (120)—The battery compartment houses the rechargeable power source that ensures the device remains operational. The compartment is designed to be easily accessible for maintenance or replacement of the battery as needed.

Portable Electronic Device (122)—The portable electronic device encompasses all the features necessary to operate the system, including the call button, speaker, power supply, and communication module. The device is lightweight and portable, making it easy to transport within the hospital environment.

Memory (124)—The memory element stores configuration data and call logs for the system. It ensures that the device can be configured to meet the specific needs of each patient and healthcare setting.

Processor (126)—The processor manages the internal operations of the device, including signal processing, communication with the communication portal, and controlling the indicator light. The processor enables the device to function efficiently and reliably.

Communication Portal (128)—The communication portal serves as the central interface where medical staff receive alerts from the wireless call buttons. It is connected to the device via wireless communication protocols, allowing real-time notification and two-way communication.

Power Source (130)—The power source provides the necessary energy to operate the wireless communication system. It may consist of a rechargeable battery, designed to provide long-lasting power, or an alternative power solution, depending on the application.

Holding Clip (132)—The holding clip is used to attach the device securely to the hospital bed, IV pole, or other suitable locations. It ensures the device remains in place during use, preventing it from being accidentally dislodged.

Rod (134)—The rod is a flexible element that can be adjusted to allow for easy positioning of the patient call button. The rod is attached to the device and can be clipped to the patient's bed, ensuring that the call button is within easy reach of the patient.

Hospital Bed (136)—The hospital bed provides the platform for attaching the device, ensuring that it is within the patient's reach. The device is mounted securely to the bed, enabling easy access to the call button.

Patient (138)—The patient interacts with the device by pressing the call button when they need assistance. The patient's comfort and ease of use are considered during the design of the system, ensuring that they can operate the device even if they have limited mobility.

Hospital/Patient System (140)—The hospital/patient system integrates the wireless communication device with the healthcare facility's central systems. It ensures that patient requests are properly routed to the appropriate staff members and that the responses are logged for future reference.

Central Database System (142)—The central database system stores and manages the data collected from patient requests, ensuring that records are kept for analysis and performance monitoring. The database may be accessed by medical staff for historical reference or to identify patterns in patient needs.

Microphone (144)—The microphone is part of the two-way communication system. It picks up the patient's voice and transmits it to the medical staff via the speaker, enabling clear and effective communication.

Audio Interface (146)—The audio interface manages the sound levels for both the speaker and microphone, ensuring that the voice transmission is clear and audible, even in noisy environments.

Radio (148)—The radio module enables wireless communication between the device and the central communication portal. Ictuses standard wireless communication protocols to ensure reliable signal transmission.

DSP (150)—The digital signal processor (DSP) manages the data signals, ensuring clear communication between the device and the healthcare staff The DSP also handles noise filtering, improving the clarity of the audio signal.

Display Indicators (152)—The display indicators provide visual feedback to the healthcare staff regarding the status of the call. They may show when a call is active or indicate the priority level of the call, such as emergency or rapid response.

There are several alternative embodiments or variations of the invention that can be used to customize the system for different healthcare settings or requirements. Some of these variations include different communication protocols, alternative materials for the device housing, and optional features such as additional sensors or environmental controls.

Communication Protocols: While the invention primarily uses wireless communication methods such as Wi-Fi or Bluetooth, alternative communication technologies such as ZigBee, Lora, or cellular networks can be used depending on the hospital's existing infrastructure and requirements. These alternative protocols may offer longer ranges or more energy-efficient communication, making them suitable for larger healthcare facilities or rural hospitals.

Alternative Housing Materials: The enclosure (104) of the device is typically made from high-quality plastic materials that are durable and easy to clean. However, other materials, such as antimicrobial coatings or medical-grade metals, may be used to enhance the device's durability and resistance to germs and contamination. These alternatives can be selected based on the specific needs of the healthcare environment.

Additional Sensors and Environmental Controls: In some variations, the wireless communication system may be equipped with additional sensors, such as temperature sensors, motion detectors, or environmental monitoring systems. These sensors can provide real-time data to the medical staff, allowing them to monitor the patient's environment and respond to changes in their condition. Additionally, the system may be equipped with environmental controls to adjust lighting or temperature in the patient's room based on their needs.

Multiple Communication Channels: The system maybe expanded to include multiple communication channels, allowing for more complex interactions between patients and medical staff for example, the system may incorporate video communication capabilities, enabling doctors to visually assess the patient's condition remotely.

The materials and methods used in the invention are critical to ensuring the device's reliability and safety in a hospital environment. The device must be durable, easy to clean, and resistant to contamination.

Materials Used for the Housing and Components: The housing (104) of the device is typically made of medical-grade plastic, such as ABS or polycarbonate, which offers strength, resistance to impact, and ease of cleaning. The plastic is treated with antimicrobial agents to reduce the risk of bacterial growth. Additionally, the material is non-porous, making it easy to wipe down with hospital-grade disinfectants. The components, such as the patient call button (106), speaker (108), microphone (144), and indicator light (102), are made from high-quality, durable materials designed to withstand the demanding environment of a hospital. The electronic components are housed in the enclosure to prevent exposure to dust, moisture, and other contaminants.

Assembly and Manufacturing Methods: The device is assembled in a controlled environment, ensuring that each component is tested and verified before being placed into the final assembly. The communication module (128) is calibrated to ensure reliable wireless communication between the device and the central communication portal (140). The power source (130) is integrated with the system to ensure the device remains operational throughout a typical hospital shift.

Quality Control: During the assembly process, each unit undergoes rigorous quality control tests to ensure functionality, durability, and compliance with medical device standards. This includes testing for signal strength, communication reliability, battery life, and ease of use.

The invention provides a reliable, easy-to-use system for improving communication and patient care in healthcare settings. Each element is designed to work together seamlessly to ensure the safety, comfort, and well-being of patients while enhancing the efficiency of healthcare providers.

While the exemplary embodiments of the present invention are described and illustrated herein, it will be appreciated that they are merely illustrative. It will be understood by those skilled in the art that various modifications in form and detail may be made therein without departing from or offending the spirit and scope of the invention as defined by the appended claims. Additionally, the invention illustratively disclosed herein suitably may be practiced in the absence of any element which is not specifically disclosed herein-and in particular, embodiment specifically contemplated, is intended to be practiced in the absence of any element which is not specifically disclosed herein.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

While this invention has been described in conjunction with the exemplary embodiments outlined above, it is evident that many alternatives, modifications and variations will be apparent to those skilled in the art. Accordingly, the exemplary embodiments of the invention, as set forth above, are intended to be illustrative, not limiting. Various changes maybe made without departing from the spirit and scope of the invention.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

What is claimed is:

1. A wireless call light device (100) for use in a hospital, comprising:
an enclosure (104) housing:
a communication portal (128) configured to place a call to a pre-programmed recipient upon activation of a patient call button (106, 110);
a speaker (108) and microphone (144) for enabling two-way audio communication between a patient (138) and hospital staff;
a memory (124) and processor (126) configured to store a user-programmable identifier corresponding to a hospital bed (136) and transmit the assigned identifier to a central database system (142) when a call is placed;
An indicator light (102) configured to illuminate when a call is initiated;
Remain illuminated until manually turned off via a staff-only reset mechanism;
Remain unaffected by repeated button presses after call initiation;
A patient call button (106) mounted on the enclosure (104) and a separately attached flexible cord button (110) with a grip (118) for patient accessibility;
A power source (130), including a hot-swappable battery housed within a battery compartment (120);
A holding clip (132) and a rod attachment mechanism (134) configured to secure the device to a hospital bed (136) or IV pole.

2. The device of claim 1, wherein the processor (126) enables a voice-based or button-based interface for assigning a bed-specific name to the device, stored in the memory (124) and transmitted via the communication portal (128).

3. The device of claim 2, wherein upon receiving a call, the recipient's device audibly announces the assigned device name (e.g., “Call from Hallway 5”), similar to caller ID functionality.

4. The device of claim 1, wherein the device confirms successful identifier assignment via an audio interface (146) by stating: “Logged in as Hallway 5.”

5. The device of claim 1, further comprising a Rapid Response button (116) configured to initiate a call to a pre-programmed emergency recipient, activate a red flashing light on the display indicators (152), require a deliberate press to prevent accidental activation, and remain illuminated until manually turned off via the staff reset button.

6. The device of claim 1, further comprising a Code Blue button (114) configured to:
Initiate a call to a pre-programmed emergency recipient with a “Code Blue” announcement.
Activate a blue flashing light on the display indicators (152).
Optionally dial a dedicated Code Blue number if programmed by hospital staff.
Require a deliberate press to prevent accidental activation.
Remain illuminated until manually turned off via the staff reset button.

7. The device of claim 6, wherein the Code Blue button (114) is programmed to notify an external hospital emergency response system via the communication portal (128).

8. The device of claim 1, wherein the patient call button (106) and wand-mounted call button (110) incorporate a resistive mechanism to prevent accidental activation.

9. The device of claim 1, further comprising a staff-only reset button, positioned separately from patient controls, allowing only hospital staff to deactivate the indicator light (102).

10. The device of claim 1, further comprising a yellow LED indicator on the display indicators (152), separately activated for low-priority notifications distinguishable from emergency alerts.

11. The device of claim 1, wherein the holding clip (132) and rod attachment mechanism (134) are adjustable to securely mount the device to various hospital equipment, including IV poles and bedside rails.

12. The device of claim 1, wherein the power source (130) includes a hot-swappable battery housed within the battery compartment (120), allowing replacement without disrupting device operation.

13. The device of claim 1, wherein the battery system includes a dedicated charging base, enabling simultaneous charging of multiple devices.

14. The device of claim 1, wherein the communication portal (128) generates an automated low-battery alert transmitted to the central database system (142).

15. The device of claim 1, further comprising a hospital/patient system (140), configured to track device location and call history in a central database system (142). Provide real-time monitoring of device status, emergency alerts, and battery levels.

16. The device of claim 15, wherein the central database system (142) generates automated reports for hospital administrators, tracking response times, patient call frequencies, and staff interactions.

17. The device of claim 1, wherein the communication portal (128) operates via Wi-Fi, Bluetooth, or a dedicated hospital network for seamless integration into existing infrastructure.

18. The device of claim 1, wherein all calls and alerts are automatically logged in the central database system (142) for compliance and review.

19. The device of claim 1, wherein the communication portal (128) includes voice-to-text transcription, enabling hospital staff to retrieve recorded messages from patients in case of missed calls.

20. A hospital-wide patient communication and assistance system, comprising:
a plurality of wireless patient communication devices (122) deployed in emergency department hallway beds (136);
a central database system (142) configured to:
receive and log call alerts from each device;
track device status and emergency notifications in real time;
provide a hospital-wide web-based dashboard for monitoring response efficiency and patient safety;
a hospital/patient system (140) allowing authorized staff to access call logs, response analytics, and device usage data.

21. A method for enabling wireless communication between a patient and hospital staff, comprising:
detecting a patient request by pressing a patient call button (106) on a wireless call light device (100);
activating an indicator light (102) upon detection of the patient request. transmitting a call signal via a communication portal (128) to a hospital/patient system (140);
establishing two-way audio communication between the patient (138) and hospital staff using a speaker (108) and a microphone (144);
maintaining the illumination of the indicator light (102) until manually turned off, irrespective of subsequent patient button presses;
preventing redundant call signals from being generated while an active call session is in progress.

22. The method of claim 21, further comprising the step of activating a code blue button (114) to transmit an emergency alert signal to the hospital/patient system (140).

23. The method of claim 21, further comprising the step of using a rapid response button (116) to generate an urgent medical alert different from an emergency alert.

24. The method of claim 21, wherein the call signal is transmitted wirelessly using a radio (148) to a central database system (142).
